(19) United States
(12) Patent Application Publication
(10) Pub. No.: US 2016/0225150 A1
(43) Pub. Date: Aug. 4, 2016

(54) Method and Apparatus for Object Distance and Size Estimation based on Calibration Data of Lens Focus

(71) Applicant: Capso Vision, Inc., Saratoga, CA (US)

(72) Inventors: Gordon C. Wilson, San Francisco, CA (US); Kang-Huai Wang, Saratoga, CA (US)

(73) Assignee: Capso Vision, Inc.

(21) Appl. No.: 15/012,840

(22) Filed: Feb. 1, 2016

Related U.S. Application Data
(60) Provisional application No. 62/110,785, filed on Feb. 2, 2015.

(51) Int. Cl. G06T 7/00; H04N 5/225; G06T 7/60; H04N 9/04; G06T 1/00; A61B 1/04; H04N 7/18

(52) U.S. Cl. CPC G06T 7/0018; A61B 1/041; H04N 5/2252; H04N 7/183; H04N 9/045; G06T 1/0007; G06T 7/0012; G06T 7/004; G06T 7/602; H04N 2005/2255; G06T 2207/10068; G06T 2207/10024; G06T 2207/30092

(57) ABSTRACT

A method for determining an object's size based on calibration data is disclosed. The calibration data is measured by capturing images with an image sensor and a lens module, having at least one objective, of the capsule camera at a plurality of object distances and/or back focal distances and deriving from the images characterizing a focus of each objective for at least one color plane. Images of lumen walls of gastrointestinal (GI) tract are captured using the capsule camera. Object distance for at least one region in the current image is estimated based on the camera calibration data and relative sharpness of the current image in at least two color planes. The size of the object is estimated based on the object distance estimated for one or more regions overlapping with an object image of the object and the size of the object image.

### CROSS REFERENCE TO RELATED APPLICATIONS

The present invention claims priority to U.S. Provisional Patent Application Ser. No. 62/110,785, filed on Feb. 2, 2015. The U.S. Provisional Patent Application is hereby incorporated by reference in its entirety.

### FIELD OF THE INVENTION

The present invention relates to in vivo capsule camera. In particular, the present invention discloses techniques for object distance and size estimation based on calibration data of lens focus.

### BACKGROUND AND RELATED ART

A technique for extending the depth of field (EDOF) of a camera and also estimating the distance of objects, captured in an image from the camera, is presented in U.S. Pat. Nos. 7,920,172 and 8,270,083 assigned to DXO Labs, Boulogne Billancourt, France. The camera uses a lens with intentional longitudinal chromatic aberration. Blue components of an image focus at shorter object distance than red components. The high-spatial-frequency information in the blue channel is used to sharpen the green and red image components for objects close to the camera. The high-spatial-frequency information in the red channel is used to sharpen the green and blue image components for objects far from the camera. The high-spatial-frequency information in the green channel is used to sharpen the blue and red image components for objects at an intermediate distance to the camera. The method works best when the color components are highly correlated, which is mostly the case in natural environments. Moreover, human visual perception is more sensitive to variations in luminance than to chrominance, and the errors produced by the technique mostly affect chrominance. The in vivo environment is a natural one and well suited for the application this technique.

By measuring the relative sharpness of each color component in a region of the image and determining quantitative metrics of sharpness for each color, the object distance may be estimated for that region of the image. Sharpness at a pixel location can be calculated based on the local gradient in each color plane, or by other standard methods. The calculation of object distance requires knowledge of how the sharpness of each color varies with object distance, which may be determined by simulation of the lens design or by measurements with built cameras.

In a fixed-focus camera, the focus is not dynamically adjusted for object distance. However, the focus may vary from lens to lens due to manufacturing variations. Typically, the lens focus is adjusted using active feedback during manufacturing by moving one or more lens groups until optimal focus is achieved. Feedback may be obtained from the image sensor in the camera module itself or from another image sensor in the production environment upon which an image of a resolution target is formed by the lens. Active alignment is a well-known technique and commonly applied. However, the cost of camera manufacturing can be reduced if it is not required. Moreover, a single lens module may hold multiple objectives, all imaging the same or different fields of view (FOVs) onto a common image sensor. Such a system is described in U.S. Pat. No. 8,717,413 assigned to Capso Vision Inc. It is used in a capsule endoscope to produce a panoramic image of the circumference of the capsule. In order for the capsule to be swallowable, the optics must be miniaturized, and such miniaturization makes it difficult to independently adjust the focus of multiple (e.g. four) lens objectives in a single module.

When applying the EDOF technique to a capsule endoscope using a lens module with multiple fixed-focus objectives, or when applying it to any imaging system with a focus that is not tightly controlled in manufacturing, a method of calibration is important to determine the focus of each objective, to store the data with an association made to the camera, and to retrieve and use the data as part of the image processing and to form an estimation of object distances from the images.

In the medical imaging applications, such as imaging the human gastrointestinal track using an in vivo camera, not only the object distance (i.e., the distance between the camera and the GI walls) but also the size of object of interest (e.g., polyps or any anomaly) is important for diagnosis. Therefore, it is very desirable to develop techniques to automatically estimate the object size using the in vivo capsule camera.

### BRIEF DESCRIPTION OF THE DRAWINGS

FIG. 1 illustrates a lens module with two objectives. For simplicity, they are shown pointing in the same direction, but they may face different directions in object space.

FIG. 2 illustrates an exemplary capsule endoscope in cross section.

FIG. 3 illustrates an exemplary flowchart for measuring calibration data, and using the data to determine an object's size according to an embodiment of the present invention.

FIG. 4 illustrates an exemplary flowchart for system incorporating an embodiment of the present invention to allow a user to measure the size of an object of interest.

### DETAILED DESCRIPTION OF THE INVENTION

In the following description, various aspects of the present invention will be described. For purposes of explanation, specific configurations and details are set forth in order to provide a thorough understanding of the present invention. Well known features may be omitted or simplified in order not to obscure the present invention.

A technique for extending the depth of field (EDOF) of a camera and also estimating the distance of objects, captured in an image from the camera, is presented in U.S. Pat. Nos. 7,920,172 and 8,270,083 assigned to DXO Labs, Boulogne Billancourt, France. The camera uses a lens with intentional longitudinal chromatic aberration. Blue components of an image focus at shorter object distance than red components. The high-spatial-frequency information in the blue channel is used to sharpen the green and red image components for objects close to the camera. The high-spatial-frequency information in the red channel is used to sharpen the green and blue image components for objects far from the camera. The high-spatial-frequency information in the green channel is used to sharpen the blue and red image components for objects at an intermediate distance to the camera. The method works best when the color components are highly correlated, which is mostly the case in natural environments. Moreover, human visual perception is more sensitive to variations in luminance than to chrominance, and the errors produced by the technique mostly affect chrominance. The in vivo environment is a natural one and well suited for the application this technique.

By measuring the relative sharpness of each color component in a region of the image and determining quantitative metrics of sharpness for each color, the object distance may be estimated for that region of the image. Sharpness at a pixel location can be calculated based on the local gradient in each color plane, or by other standard methods. The calculation of object distance requires knowledge of how the sharpness of each color varies with object distance, which may be determined by simulation of the lens design or by measurements with built cameras. In a fixed-focus camera, the focus is not dynamically adjusted for object distance. However, the focus may vary from lens to lens due to manufacturing variations. Typically, the lens focus is adjusted using active feedback during manufacturing by moving one or more lens groups until optimal focus is achieved. Feedback may be obtained from the image sensor in the camera module itself or from another image sensor in the production environment upon which an image of a resolution target is formed by the lens. Active alignment is a well-known technique and commonly applied. However, the cost of camera manufacturing can be reduced if it is not required. Moreover, a single lens module may hold multiple objectives, all imaging the same or different fields of view (FOVs) onto a common image sensor. Such a system is described in U.S. Pat. No. 8,717,413 assigned to Capso Vision. It is used in a capsule endoscope to produce a panoramic image of the circumference of the capsule. In order for the capsule to be swallowable, the optics must be miniaturized, and such miniaturization makes it difficult to independently adjust the focus of multiple (e.g. four) lens objectives in a single module. When applying the EDOF technique to a capsule endoscope using a lens module with multiple fixed-focus objectives, or when applying it to any imaging system with a focus that is not tightly controlled in manufacturing, a method of calibration is important to determine the focus of each objective, to store the data with an association made to the camera, and to retrieve and use the data as part of the image processing and to form an estimation of object distances from the images.

Knowledge of object distance is valuable in a number of ways. First, it makes it possible to determine the size of objects based on the image height of the object. In the field of endoscopy, the clinical significance of lesions such as polyps in the colon is partly determined by their size. Polyps larger than 10 mm are considered clinically significant and polyps larger than 6 mm generally are removed during colonoscopy. These size criteria are provided as examples, but other criteria may be used, depending on clinical practice. Colonoscopists often use a physical measurement tool to determine polyp size. However, such a tool is not available during capsule endoscopy. The size must be estimated based on images of the polyp and surround organ alone, without a reference object. The EDOF technique allows the distance of the polyp from the capsule to be estimated and then the diameter or other size metric can be determined based on the size of the poly in the image (image height).

The physician typically views the video captured by the capsule on a computer workstation. The graphical user interface (GUI) of the application software includes a tool for marking points on the image, for example by moving a cursor on the display with a mouse and clicking the mouse button when the cursor is at significant locations, such as on two opposing edges of the polyp. The distance between two such marks is proportional to the diameter. The physician could also use the mouse to draw a curve around the polyp to determine the length of its perimeter. Similar functions can be performed by arrow keys to move the cursor. Also, image processing algorithms can be used to determine the lesion size automatically. The physician could indicate the location of the lesion to the software, for example by mouse-clicking on it using the GUI. Then routines such as edge-detection would be used to identify the perimeter of the polyp or other lesion. The program than determines size parameters such as diameter, radius, or circumference based on the size of the object's image, measured in pixels, and the estimated object distance for the lesion using the EDOF technique as described in U.S. Pat. No. 7,920,172. The software may use algorithms to identify lesions automatically, for example using algorithms based on machine learning, and then measure their size. The user of the software might then confirm the identifications made automatically by the analysis of the video by the software. This method of determining object size can be applied to a wide variety of objects and features both in vivo and ex vivo in various applications and fields of practice.

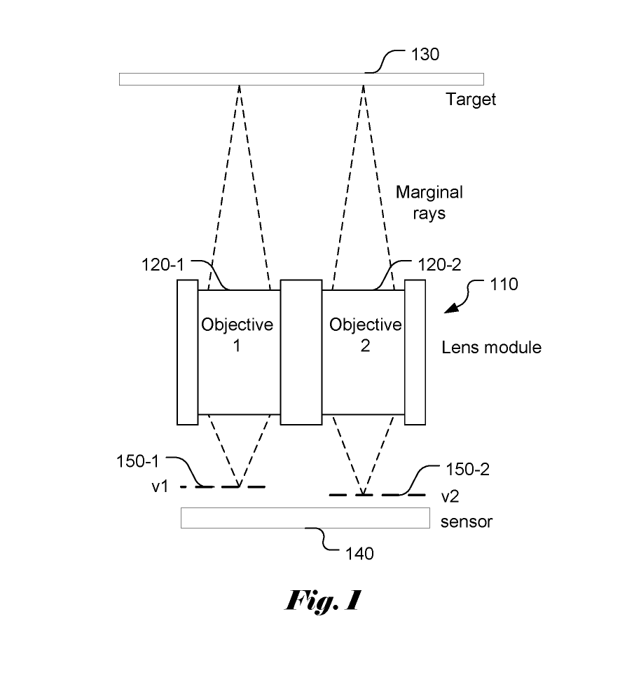

The measurement of the lens focus can occur during or after lens assembly or after camera assembly. FIG. 1 illustrates a lens module 110 with two objectives (120-1 and 120-2). For simplicity, they are shown pointing in the same direction, but they may face different directions in object space. The resolution of the lens is tested by placing one or more resolution targets (130), which may comprise patterns with contrast such as edges and lines, in front of each objective. An image sensor (140) is placed in image space. The sensor captures images of the target imaged through the objectives. The spatial frequency response (SFR), contrast transfer function (CTF), modulation transfer function (MTF) or other measure of “sharpness” can be determined from the sensor-captured image. The position of the sensor can be moved longitudinally to measure the sharpness as a function of back focal distance (e.g. a “through-focus MTF”). Thus, the image plane V1 (150-1) can be determined for objective 1 and v2 (150-2) for objective 2. Instead of moving the sensor through the image plane, another relay lens may be used to create an image of the sensor which is moved through the objective image plane. Similarly, the target may be a physical target or a projection of a target to the same position.

Finite conjugate lenses, such as those used in capsule endoscopy, can be characterized by changing the distance from the target (or projection of a target) to the lens module instead of moving the sensor. Either way, the back focal length of each objective can be measured. The back focal distance (BFD) is the distance from a reference plane on the lens module to the image plane of an objective in the module for a fixed object distance. As the object distance is varied, the BFD varies.

If the lens is designed to have chromatic aberration, then the BFD varies with the wavelength of light. The lens test may be performed with illumination limited to a particular wavelength band. Measurements might be made with multiple illumination wavelength bands to characterize the variation in BFD with wavelength. The sensor has color filters that restrict the wavelength band for sets of pixels arrayed on the sensor, for example in a Bayer pattern. Thus, white light illumination may be used, and the sharpness can be measured for red, green, and blue pixels (i.e. pixels covered with colored filters that pas red, green, and blue light respectively). BFDs can be determined for each color. The sensor may have pixels with color filters at other colors besides or in addition to the standard red, blue, and green, such as yellow, violet, or infrared or ultraviolet bands of wavelengths.

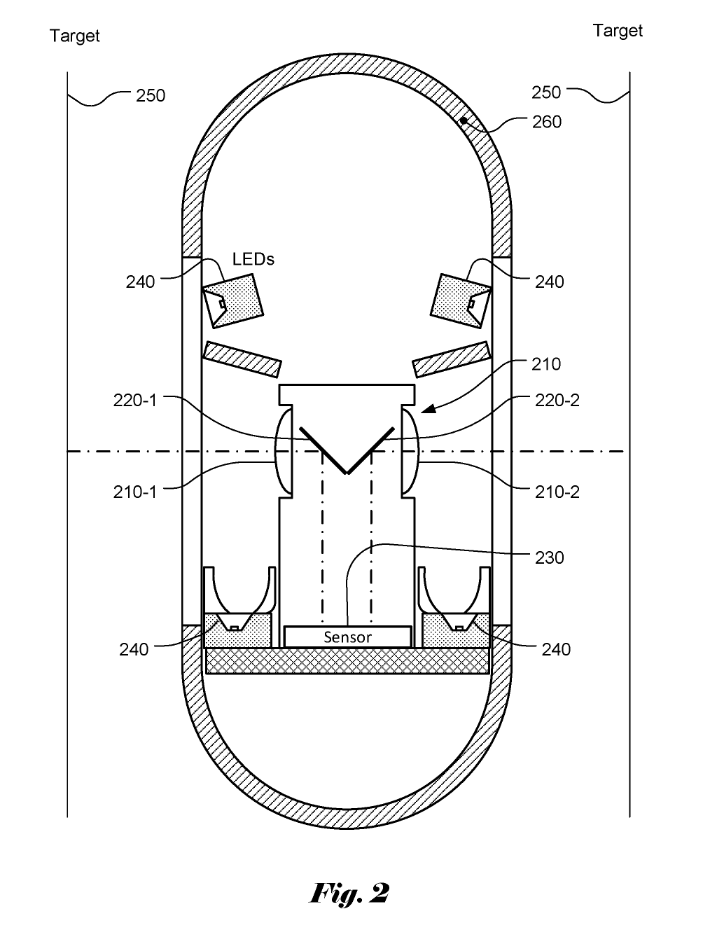

The lens focus can also be determined after the camera is assembled. FIG. 2 shows a capsule endoscope in cross section. The lens module (210) has two objectives (210-1 and 210-2) shown, although it typically has four with angular spacing of 90 degrees. Each objective has a fold mirror (220-1 and 220-2) that folds the optical axis (shown by dashed lines) from a lateral to longitudinal direction, and each optical axis intersects the image sensor (230) which is ideally located at the back focal plane of all the objectives. The capsule camera also includes multiple LEDs (240) to illuminate the target (250). The lens module (210), fold mirrors (220-1 and 220-2), image sensor (230) and LEDs (240) are enclosed in a sealed capsule housing (260). Due to manufacturing variation, the intersection of each optical axis and the image sensor may not be exactly for each objective. The focus error is characterized by moving the targets (or projections thereof) and capturing images at multiple object distances with the camera. For each objective and color the image will be sharpest for a particular object distance. For the ith objective the optimal object distance is u_opt_i. u_opt_i is directly related to the BFD at fixed object distance. Measuring one allows the other to be determined. Both are a function of wavelength. u_opt_i may be measured as a function of wavelength and/or sensor color plane.

The calibration data on the lens module in the camera must be stored and associated with the camera for future use in processing and analyzing images captured with the camera. The calibration data may be stored in non-volatile memory in the capsule system or it may be stored on a network server labelled with a serial number or other identifier linking it to the camera.

When the camera is in use, images are captured and stored. They may be stored in the capsule and also transferred from the capsule to an external storage medium such as a computer hard drive or flash memory. The calibration data are retrieved from the storage in the camera or from the network storage. The images are analyzed and processed in the camera, in an external computer, or in a combination of the two, using the calibration data. Methods for capturing, storing, and using camera calibration data were described in U.S. Pat. No. 8,405,711, assigned to Capso Vision Inc.

Assume that u_opt_i corresponds to the object distance for the green channel with the best focus for the camera assembled with the sensor at fixed object distance. By measuring the sharpness of the red, green, and blue channel, we can determine the object distance of an object capture in the image relative to u_opt_i. The object distance is a function of the sharpness of the red, blue, and green channels, u_opt_i calibration for each color, and possibly other camera calibration parameters and measured data such as temperature. This function describes a model which may be based on simulation, theory, or empirical measurements or a combination thereof. Normally, the amount of chromatic aberration will not vary from lens to lens much. Thus, it may be adequate to only measure and store focus calibration data that allows for the calculation of u_opt_i for only one color, e.g. green.

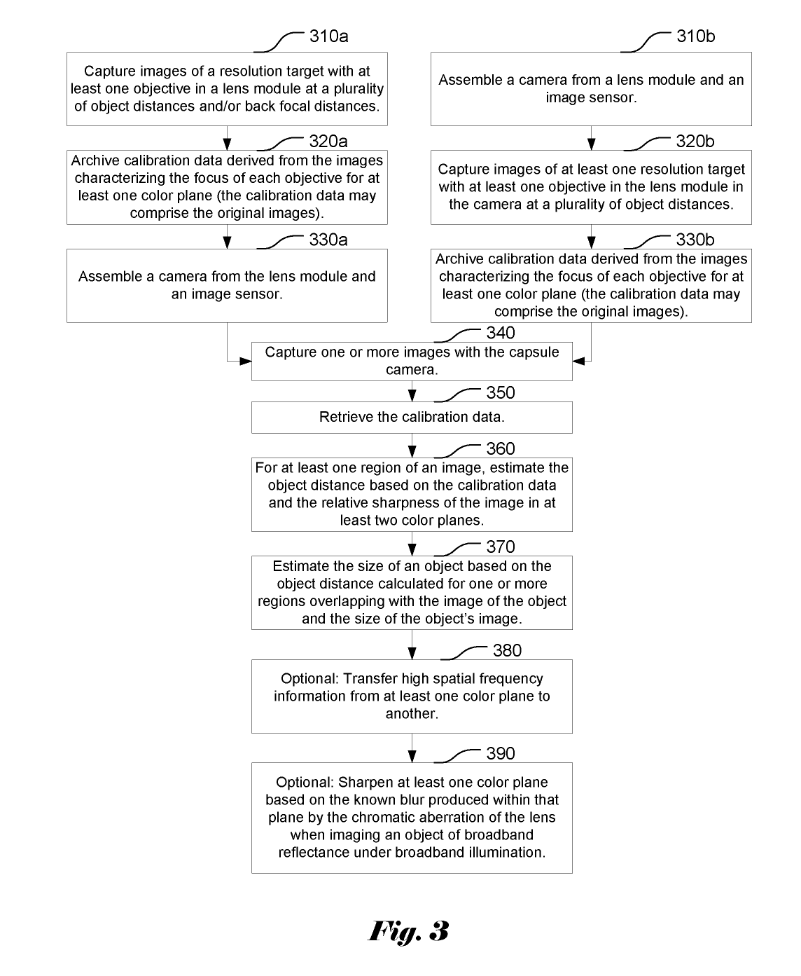

The method for measuring calibration data and using the data to determine an object's size is shown in FIG. 3. The method may include the steps of “extending the depth of field” (i.e. using high frequency information from a sharp color plane to sharpen at least one other color plane). Chromatic aberration will produce some blurring of the image within each color plane as well as across color planes since each color plane includes a range of wavelengths passed by the color filter in the color filter array on the sensor. The amount of blur depends on the spectrum of light which passes through the filter, which is dependent on the spectrum of the filter, of the illumination source, and of the reflectance of the object. Statistically, the blur will be constant enough that it can be reduced by methods such as deconvolution.

In FIG. 3, the branch on the left (steps 310a, 320a and 330a) corresponds to capturing the calibration data before assembling the camera from the lens module and the image sensor, while the branch on the right (steps 310b, 320b and 330b) corresponds to capturing the calibration data after assembling the camera from the lens module and the image sensor. These steps are performance individually for each capsule camera before the capsule camera is used in vivo to capture images for diagnosis. In step 310a, images of a resolution target with at least one objective in a lens module at a plurality of object distances and/or back focal distances are captured. In step 320a, the calibration data derived from the images characterizing the focus of each objective for at least one color plane (the calibration data may comprise the original images) are archived. As mentioned before, the calibration data can be archived either outside the capsule camera or inside the capsule camera. The capsule camera is then assembled and ready for use as shown in step 330a. The branch on the right hand side comprises the same steps as the branch on the left at different order.

In FIG. 3, steps 340 through 370 correspond to the process for image capture and object distance/size estimation using the calibration data. In step 340, one or more images are captured using the capsule camera. The calibration data is retrieved in step 350. For at least one region of an image, the object distance is estimated based on the calibration data and the relative sharpness of the image in at least two color planes in step 360. The size of an object is then estimated based on the object distance calculated for one or more regions overlapping with the image of the object and the size of the object's image in step 370. The calibration data may be stored inside or outside the capsule camera. Furthermore, the steps 350 through 370 may be performed outside the capsule camera using an image viewing/processing device, such as a personal computer, a mobile device or a workstation. Furthermore, if desired, optional steps 380 and 390 may be performed to improve image quality. In step 380, high spatial frequency information is transferred from at least one color plane to another. In step 390, at least one color plane is sharpened based on the known blur produced within that plane by the chromatic aberration of the lens when imaging an object of broadband reflectance under broadband illumination.

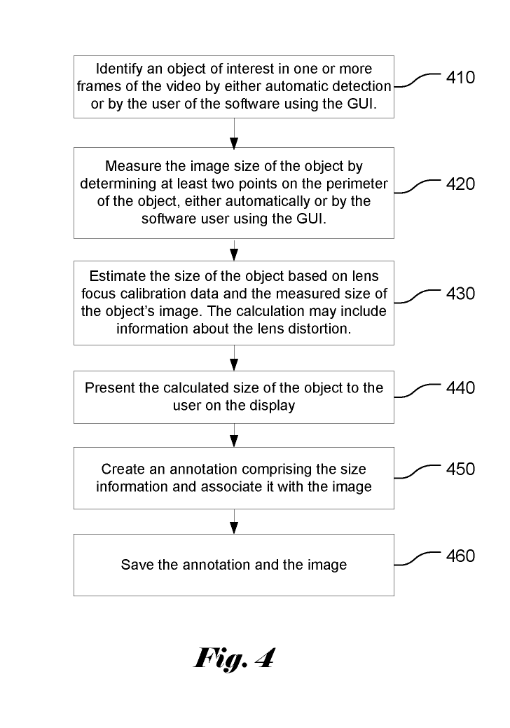

FIG. 4 illustrates an exemplary flowchart for system incorporating an embodiment of the present invention to allow a user to measure the size of an object of interest. In this example, an object of interest in one or more frames of the video can be identified by either automatic detection or by the user of the software using the GUI in step 410. The image size of the object can be measured by determining at least two points on the perimeter of the object either automatically or by the software user using the GUI in step 420. The size of the object can be estimated based on lens focus calibration data and the measured size of the object's image in step 430. The calculation may include information about the lens distortion. The calculated size of the object can be presented to the user on the display in step 440. The user may create an annotation comprising the size information and associate it with the image (step 450) and save the annotation and the image (step 450).

The flowcharts shown are intended to illustrate examples of object distance/size estimation using camera calibration data according to the present invention. A person skilled in the art may modify each step, re-arranges the steps, split a step, or combine steps to practice the present invention without departing from the spirit of the present invention.

The object height h is the image height h′ times the magnification m. The magnification is inversely proportional to the object distance u, m(x,y)=k(x,y)/u. Due to lens distortion k is a function of pixel position (x,y) in the image. The object height is thus given by

h=(1/u)∫k(x, y)dl

where the integration is along a line segment from one side of the object image to the other. The lens distortion is relatively constant for a given design, but it too may be calibrated in manufacturing and the calibration data stored with the focus calibration data.

The invention may be embodied in other specific forms without departing from its spirit or essential characteristics. The described examples are to be considered in all respects only as illustrative and not restrictive. Therefore, the scope of the invention is indicated by the appended claims rather than by the foregoing description. All changes which come within the meaning and range of equivalency of the claims are to be embraced within their scope.

1. A method for determining an object size from one or more images of an object using camera calibration data, wherein said one or more images of the object captured by a capsule camera, comprising:
receiving camera calibration data corresponding to a capsule camera, wherein the camera calibration data is measured by capturing images with an image sensor and a lens module, having at least one objective, of the capsule camera at a plurality of object distances and/or back focal distances and deriving from the images characterizing a focus of each objective for at least one color plane;
capturing one or more current images of lumen walls of gastrointestinal (GI) tract using the capsule camera;
estimating object distance for at least one region in the current image based on the camera calibration data and relative sharpness of the current image in at least two color planes; and
estimating a size of the object based on the object distance estimated for one or more regions overlapping with an object image of the object and the size of the object image.
